(19) United States
(12) Patent Application Publication
(10) Pub. No.: US 2024/0341509 A1
(43) Pub. Date: Oct. 17, 2024

(54) PADDED SLEEVES

(71) Applicant: SLEEPY SLEEVES LLC, Chicago, IL (US)

(72) Inventors: Zachary A. Rubin, Chicago, IL (US); Jay G. Perlman, Gandia (ES)

(21) Appl. No.: 18/683,146

(22) PCT Filed: Aug. 15, 2022
(86) PCT No.: PCT/US2022/040351

§ 371 Date: Feb. 12, 2024

Related U.S. Application Data
(60) Provisional application No. 63/233,314, filed on Aug. 15, 2021.

(51) Int. Cl. A47G 9/10; A41D 13/08; A41D 20/00; A47G 9/00

(52) U.S. Cl. CPC A47G 9/1045; A41D 13/08; A41D 20/00; A47G 9/1027; A47G 2009/003

(57) ABSTRACT

The present disclosure provides padded sleeves, such as sleeves including a pillow-type padded area convenient for use by travelers, and methods of making and using same.

### PRIORITY CLAIM

This application claims priority to U.S. Provisional Patent Application Ser. No. 63/233,314, filed on Aug. 15, 2021, the entire contents of which are incorporated herein by reference and relied upon.

### FIELD OF DISCLOSURE

The present disclosure relates to padded sleeves, such as sleeves including a pillow-type padded area convenient for use by travelers.

### BACKGROUND

Currently available travel pillows are bulky and prone to slippage during use. There remains a need for travel-type pillows that are convenient to use and carry. The present disclosure describes products and methods of using such products that meet that need.

### SUMMARY

The present disclosure relates to padded sleeves, such as sleeves including a pillow-type padded area convenient for use by travelers.

In some embodiments, the present disclosure provides a wearable arm sleeve comprising: a flexible sleeve including a bicep end and a cuff end disposed opposite the bicep end; and padding disposed on said flexible sleeve and covered by an outer shell having an outer surface.

In some embodiments, the present disclosure provides a wearable head sleeve comprising: a flexible sleeve including a neck end and a crown end disposed opposite the neck end; and padding disposed on said flexible sleeve and covered by an outer shell having an outer surface.

In other embodiments, the present disclosure provides a wearable pillow comprising: a tube-shaped body configured for placement about a portion of a user's body; and a padding compartment disposed on at least a portion of the tube-shaped body.

These and other embodiments are described in greater detail herein.

### BRIEF DESCRIPTION OF THE DRAWINGS

The detailed description of some embodiments of the present disclosure is made below with reference to the accompanying figures, wherein like numerals represent corresponding parts of the figures.

FIG. 1 shows a front plan view of a padded sleeve consistent with one embodiment of the present disclosure.

FIG. 2 shows a rear plan view of the padded sleeve of FIG. 1.

FIG. 3 shows a rendering of a padded sleeve consistent with another embodiment of the present disclosure.

FIG. 4 shows a photograph of a padded sleeve consistent with another embodiment of the present disclosure.

FIG. 5 shows a photograph of the padded sleeve of FIG. 4.

FIG. 6 shows a photograph of the padded sleeve of FIG. 4.

FIG. 7 shows a photograph of a padded sleeve consistent with another embodiment of the present disclosure.

FIG. 8 shows a photograph of the padded sleeve of FIG. 7.

FIG. 9 shows a photograph of a padded sleeve consistent with another embodiment of the present disclosure.

FIG. 10 shows a photograph of the padded sleeve of FIG. 9.

FIG. 11 shows a photograph of a padded sleeve consistent with another embodiment of the present disclosure.

FIG. 12A shows a photograph of the padded sleeve of FIG. 11 in an inside-out configuration.

FIG. 12B shows a rear perspective photograph of the padded sleeve of FIG. 11

FIG. 13 shows a photograph of the padded sleeve of FIG. 11 worn about the head of a user.

FIG. 14 shows a side view photograph of the padded sleeve of FIG. 11 worn about the head of a user.

FIG. 15 shows a side view photograph of the padded sleeve of FIG. 11 worn about the head of a user.

FIG. 16 shows a from perspective photograph of the padded sleeve of FIG. 11 worn about the head of a user.

FIG. 17 shows a photograph of a padded sleeve consistent with one embodiment of the present disclosure.

FIG. 18 shows a photograph of a padded sleeve consistent with the embodiment of FIG. 17 worn about the arm of a user.

FIG. 19 shows a photograph of a padded sleeve consistent with the embodiment of FIG. 17 worn about the arm of a user and in use to support the head of the user.

These and other embodiments are described more fully herein below.

### DETAILED DESCRIPTION

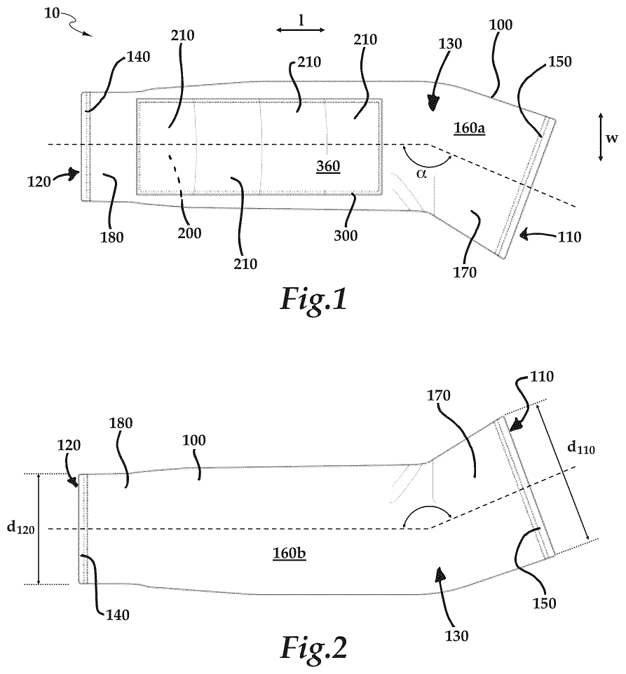

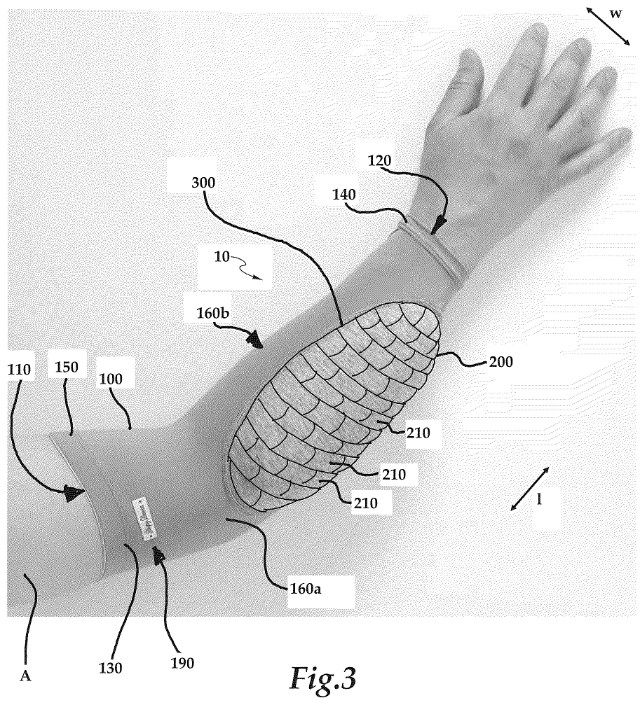

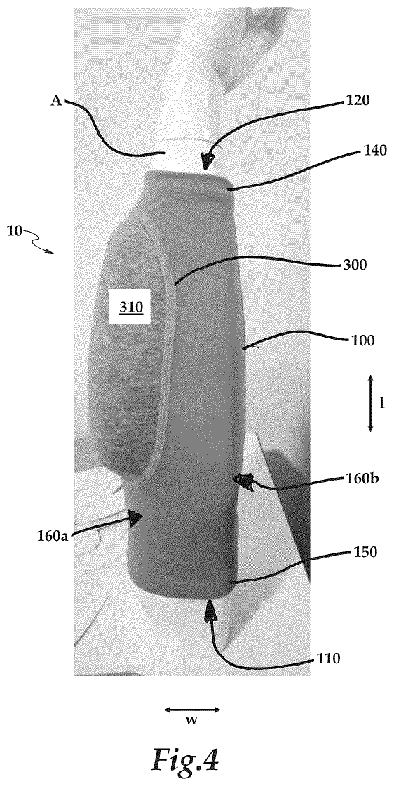

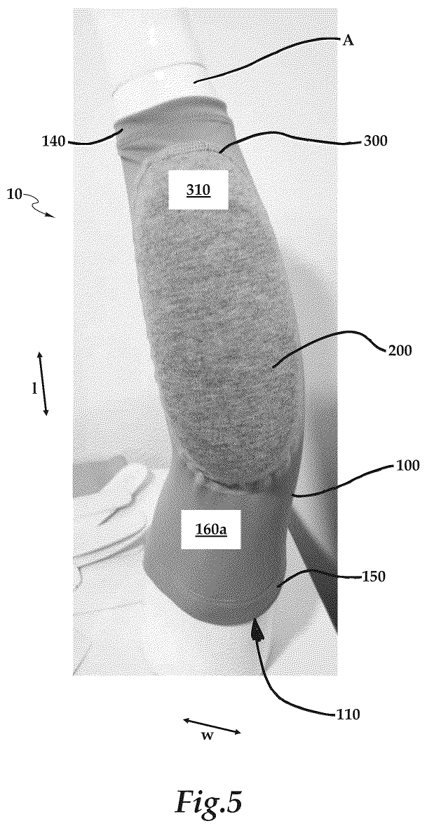

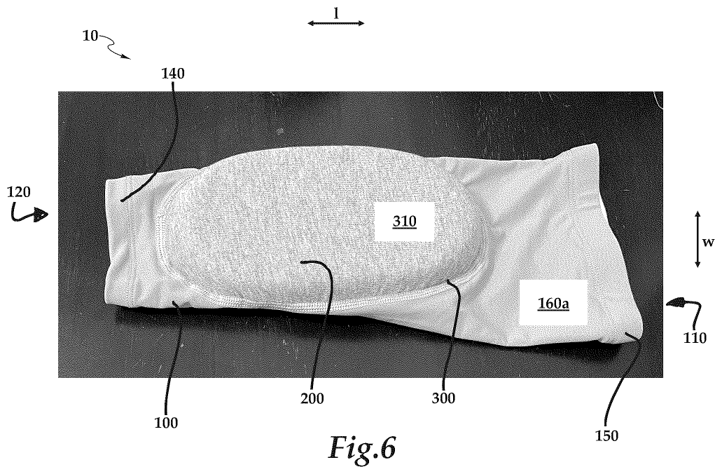

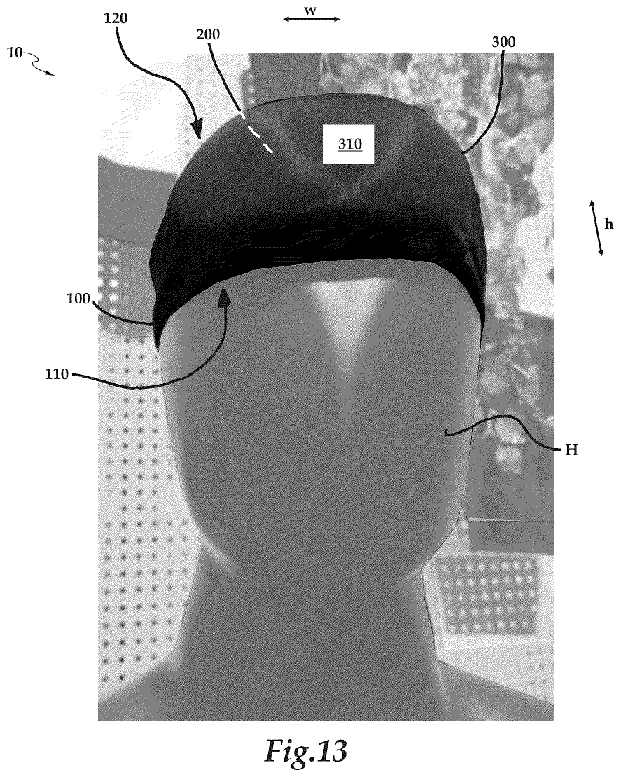

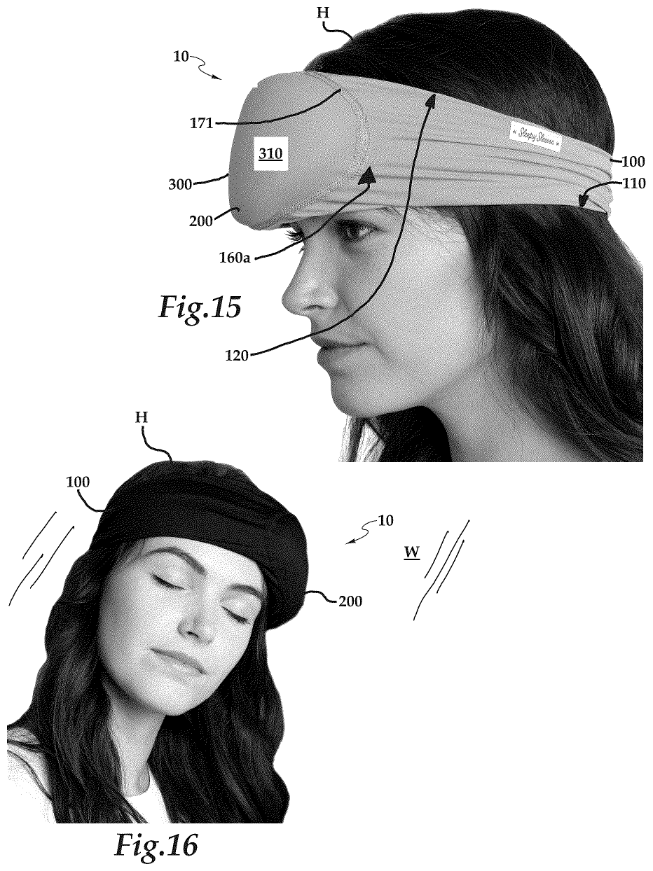

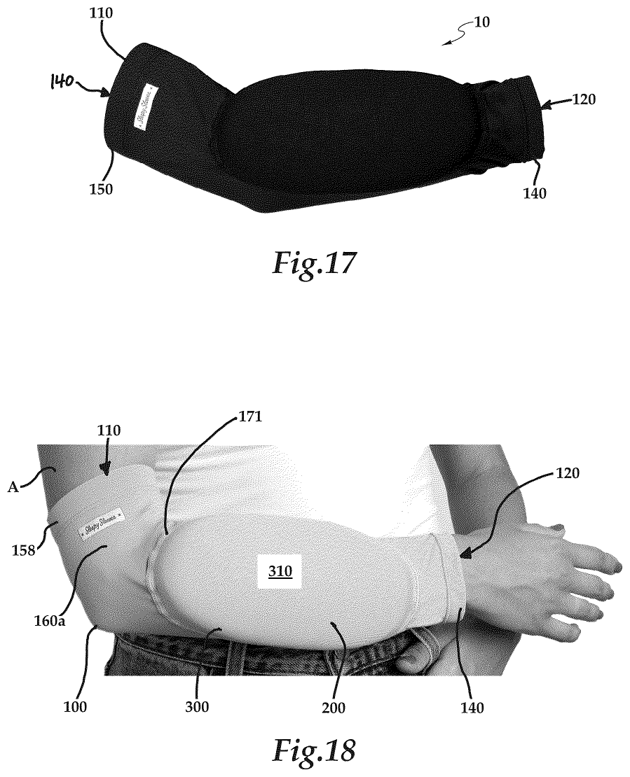

Referring generally to FIGS. 1-19, the present disclosure provides padded sleeves 10, such as wearable padded sleeves 10 configured to be worn on an arm A, a leg L, or a head H of a user.

Referring now specifically to FIGS. 1-6 and 17-19, wearable sleeves 10 generally configured to be worn on an arm A or leg L of a user generally include a flexible sleeve 100 and padding 200 disposed on the flexible sleeve 100.

The flexible sleeve 100 includes a bicep end 110 and a cuff end 120 opposite the bicep end 120. The flexible sleeve 100 is generally tubular in shape, with an outer surface 160 and an inner surface (not shown) opposite the outer surface 160. In use, the inner surface contacts the user's arm or leg skin, while the outer surface 160 is generally not in contact with the user's arm or leg skin. In some embodiments, the tubular shape of the flexible sleeve 100 is permanent; for example and without limitation, the tubular shape may be formed from one or more sheets of material that are stitched or bonded together to form a tubular shape.

In other embodiments, the tubular shape is formable and releasable by the user. For example and without limitation, flexible sleeves 100 of such embodiments may include a first mating feature disposed near one edge of the sheet of material, and a second mating feature disposed on the sheet of material remotely from the first mating feature and configured to releasably mate with the first mating feature to form the tubular shape. The first mating feature may be a magnet, a snap stud, a snap socket, a button, a button hole, hook fastener, loop fastener, or one half of a zipper; while the second mating feature may be complementary to (e.g., releasably mates with) the first mating feature. The second mating feature may therefore be a magnet, a snap socket, a snap stud, a button hole, a button, loop fastener, hook fastener, or the other half of a zipper. In some embodiments, the first mating feature is one half of a hook-and-loop fastener (e.g., VELCRO), while the second mating feature is the other complimentary half of the hook-and-loop fastener.

For embodiments configured to be worn on an arm A, the bicep end 110 may have an inner diameter d₁₁₀ that is relatively larger than the inner diameter d₁₂₀ of the cuff end 120.

For embodiments configured to be worn on a leg L, the bicep end 110 (e.g., the end worn closer to the hip joint of the leg L) may have an inner diameter that is relatively larger than the inner diameter of the cuff end 120 (e.g., the end worn farther from the hip joint).

The flexible sleeve 100 may have a length sufficient to cover all or substantially all of the user's forearm. In some embodiments, the flexible sleeve 100 has a length sufficient to cover all or substantially all of the user's forearm and at least a portion of the user's upper arm.

The flexible sleeve 100 may be tapered from the generally larger bicep end 110 to the generally narrower cuff end 120. In some embodiments, the taper is generally linear (e.g., the diameter d of the flexible sleeve tapers consistently from a bicep end diameter d₁₁₀ at the bicep end 110 to a smaller cuff end diameter d₁₂₀ at the cuff end 120 as a function of the distance from the bicep end 110). In other embodiments, the flexible sleeve 100 includes two differently tapered sections. For example and without limitation, the flexible sleeve 100 may have a first, relatively steeper taper from the bicep end 110 to the elbow region 130, and a second, relatively shallower taper from the elbow region 130 to the cuff end 120.

In some embodiments, the flexible sleeve 100 includes an upper arm section 170 and a lower arm section 180. In some embodiments, the upper arm section 170 is disposed at an angle α relative to the lower arm section 180. In some embodiments, the angle α is obtuse. In some embodiments, the angle α is about 135° to about 175°, for example about 135°, about 136°, about 137°, about 138°, about 139º, about 140°, about 141º, about 142°, about 143º, about 144°, about 145°, about 146°, about 147°, about 148°, about 149°, about 150°, about 151º, about 152°, about 153°, about 154°, about 155°, about 156°, about 157°, about 158°, about 159°, about 160°, about 161°, about 162°, about 163°, about 164°, about 165°, about 166°, about 167º, about 168°, about 169°, about 170°, about 171°, about 172°, about 173°, about 174°, or about 175°. In some embodiments, the angle α is about 165° to about 175°, or about 170°.

In some embodiments, a hem 140 is disposed near the cuff end 120 of the flexible sleeve 100. When present, the hem 140 improves durability of the flexible sleeve 100 by, for example, reducing, preventing or truncating unraveling of the cuff end 120 of the flexible sleeve 100.

In some embodiments, a hem 150 is disposed near the bicep end 110 of the flexible sleeve 100. When present, the hem 150 improves durability of the flexible sleeve 100 by, for example, reducing, preventing or truncating unraveling of the bicep end 110 of the flexible sleeve 100.

In some embodiments, the outer surface 160 of the flexible sleeve 100 includes a graphic design 190, a logo, or a designated space for a graphic design or logo.

The flexible sleeve 100 may comprise, consist essentially of, or consist of any suitable material. In some embodiments, the flexible sleeve 100 comprises, consists essentially of, or consists of a stretchable fabric. In some embodiments, the stretchable fabric has a lengthwise stretchability (e.g., is stretchable along the length/of the flexible sleeve 100) of about 5% to less than 30%. For example and without limitation, the lengthwise stretchability may be about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or less than 30%.

In some embodiments, the stretchable fabric has a width-wise stretchability (e.g., is stretchable along the width w of the flexible sleeve 100) of about 5% to less than 45%. For example and without limitation, the width-wise stretchability may be about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or less than 45%.

In some embodiments, the size of the flexible sleeve 100 is only adjustable due to the stretchability of the flexible sleeve 100 material. In such embodiments, the flexible sleeve 100 does not include any size adjustment straps or other tensioning features other than the stretchable properties of the material comprising the flexible sleeve 100.

In some embodiments, the flexible sleeve 100 is a unitary piece of material.

In some embodiments, the flexible sleeve 100 does not include transversal folding lines or creases.

In some embodiments, the flexible sleeve 100 is a compression sleeve.

In some embodiments, the flexible sleeve 100 comprises, consists essentially of, or consists of cotton, nylon, rayon, polyester, mesh, fishnet, lycra, jersey material, jersey material including microholes, denim, leather, vinyl, or a combination of any two or more of the foregoing.

In some embodiments, the flexible sleeve 100 includes at least one inflexible region (e.g., rigid or substantially rigid). In such embodiments, the inflexible region may comprise, consist essentially of, or consist of any suitable rigid or substantially rigid material, such as plastic, steel, aluminum, copper, zinc, carbon fiber, fiberglass, or any two or more of the foregoing.

In some embodiments, the flexible sleeve 100 includes at least one opening in the flexible sleeve configured to promote air circulation against skin of a user.

The padding 200 is generally disposed on a lateral side of the flexible sleeve 100. For example and without limitation, the padding 200 may be disposed on the lateral forearm surface 160a of the flexible sleeve 100 in embodiments wherein the wearable pillow 10 is configured to be worn on an arm A. In such embodiments, the medial forearm surface 160b may optionally include padding 200, or may lack padding.

The padding 200 may in some embodiments be covered by an outer shell 300 that includes an outer surface 310. The outer shell 300 is secured to the flexible sleeve 100, for example by stitching, adhesive, fasteners, or a combination thereof. In other embodiments, the padding 200 is disposed in a cavity of the flexible sleeve 100, for example in a cavity formed by two or more layers of the flexible sleeve 100. The outer shell 300 may be formed of the same material(s) as the flexible sleeve 100. Alternatively, the outer shell 300 may comprise, consist essentially of, or consist of a material different than the flexible sleeve 100. In some embodiments, the outer shell 300 comprises, consists essentially of, or consists of a soft material, such as cotton, jersey, felt, fleece, jersey with microholes, or any two or more of the foregoing.

The padding 200 may have any suitable thickness to provide a level of comfort to the user, for example when the head H is rested upon the padding 200. In some embodiments, the padding 200 has a thickness sufficient to provide at least 1 cm of compressed padding between the user's head H and the body part (e.g., arm, leg, etc.) upon which the device 10 is worn, or between the user's head H and the surface upon which the user rests its head H when the device 10 is worn on the user's head H. In some embodiments, the padding 200 has a thickness (e.g., uncompressed thickness) of about 1 cm to about 6 cm, for example about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2 cm, about 2.1 cm, about 2.2 cm, about 2.3 cm, about 2.4 cm, about 2.5 cm, about 2.6 cm, about 2.7 cm, about 2.8 cm, about 2.9 cm, about 3 cm, about 3.1 cm, about 3.2 cm, about 3.3 cm, about 3.4 cm, about 3.5 cm, about 3.6 cm, about 3.7 cm, about 3.8 cm, about 3.9 cm, about 4 cm, about 4.1 cm, about 4.2 cm, about 4.3 cm, about 4.4 cm, about 4.5 cm, about 4.6 cm, about 4.7 cm, about 4.8 cm, about 4.9 cm, about 5 cm, about 5.1 cm, about 5.2 cm, about 5.3 cm, about 5.4 cm, about 5.5 cm, about 5.6 cm, about 5.7 cm, about 5.8 cm, about 5.9 cm, about 6 cm. In some embodiments, the padding 200 has a minimum compressed thickness (e.g., when the user rests its head H upon the padding 200) of not less than about 1 cm, for example not less than about 1 cm, not less than about 2 cm, not less than about 3 cm, not less than about 4 cm, not less than about 5 cm, or not less than about 6 cm.

In some embodiments, the padding 200 has a generally convex shape. For example and without limitation, the padding 200 may have a convex shape that includes a convex surface contour that bows away from the outer surface 160 of the flexible sleeve 100. In other embodiments, the padding 200 is not convex. For example and without limitation, the padding 200 in such alternative embodiments may have a flat outer contour (e.g., generally parallel with the outer surface 160 of the flexible sleeve 100) or a concave outer contour (e.g., bows inwardly towards the outer surface 160 of the flexible sleeve 100).

The padding 200 may extend substantially the length of the flexible sleeve 100. In other embodiments, the padding 200 extends only a portion of the length of the flexible sleeve 100. In such embodiments, the padding may be disposed about 1 cm to about 10 cm from the cuff end 120, for example about 1 cm, about 2 cm, about 3 cm, about 4 cm, about 5 cm, about 6 cm, about 7 cm, about 8 cm, about 9 cm, or about 10 cm from the cuff end 120.

In some embodiments, the padding 200 may be disposed about 1 cm to about 18 cm from the bicep end 110. For example and without limitation, the padding 200 may be disposed about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, or about 18 cm from the bicep end 110.

The padding 200 may, in some embodiments, be disposed over all or substantially all of the outer surface area of the flexible sleeve 100. In other embodiments, the padding 200 is disposed over only a fraction of the outer surface area of the flexible sleeve 100, such as not more than about 95% of the outer surface area of the flexible sleeve 100. In some embodiments, the padding 200 is disposed over not more than about 95%, not more than about 90%, not more than about 85%, not more than about 80%, not more than about 75%, not more than about 70%, not more than about 65%, not more than about 60%, not more than about 55%, not more than about 50%, not more than about 45%, not more than about 40%, not more than about 35%, not more than about 30%, not more than about 25%, not more than about 20%, not more than about 15%, not more than about 10%, or not more than about 5% of the outer surface area of the flexible sleeve 100.

In some embodiments, the padding 200 is disposed over about 10 cm² of the outer surface of the flexible sleeve 100. In other embodiments, the padding 200 is disposed over more than about 10 cm² of the outer surface of the flexible sleeve 100, for example about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm², about 130 cm², about 135 cm², about 140 cm², about 145 cm², about 150 cm², about 155 cm², about 160 cm², about 165 cm², about 170 cm², about 175 cm², about 180 cm², about 185 cm², about 190 cm², about 195 cm², or about 200 cm² of the outer surface of the flexible sleeve 100.

In some embodiments, the padding 200 does not include any transversal folding lines (e.g., separations between padded cells 210) that enable folding of the padded sleeve 10 on itself for compact storage.

In some embodiments, the padding 200 is disposed only on a portion of the flexible sleeve 100 corresponding to the lower portion of the arm A (e.g., the forearm); in such embodiments no padding 200 is disposed on a portion of the flexible sleeve 100 corresponding to the upper portion of the arm A (e.g., the bicep or triceps areas).

In some embodiments, the padding 200 is configured to not be removed from the padded sleeve 10.

In some embodiments, the padding 200 is disposed only on the outer surface of the flexible sleeve 100; in such embodiments no padding 200 is disposed on the inner surface of the flexible sleeve 100 (e.g., in direct contact with the user's arm A).

The padding 200 may comprise, consist essentially of, or consist of any suitable padding material that offers comfort and support to a user, for example when the user's head H is rested upon the padding 200. In some embodiments, the padding 200 comprises, consists essentially of, or consists of cotton, down, microbeads, gel, fluid, expanded foam, open-cell foam, closed-cell foam, a polymer (e.g., an elastic polymer), a polyfoam, or any two or more of the foregoing. In some embodiments, the padding 200 includes a foam, such as viscoelastic polyurethane foam.

In some embodiments, the padding 200 comprises, consists essentially of, or consists of an inflatable bladder. The inflatable bladder may include a valve (e.g., an inflation port) for selectively inflating and deflating the inflatable bladder.

In some embodiments, the padding 200 is a unitary piece of padding. In other embodiments, the padding comprises a plurality of segments or cells 210. For example and without limitation, the padding may include 2 to about 100 cells 210, or more than 100 cells 210. Each cell 210 may have a polygon shape, such as a circle, an oval, a rectangle (e.g., a square), a pentagon, a hexagon, a heptagon, an octagon, etc. For example, the cells 210 depicted representatively in FIG. 3 each have a generally rectangular shape. The cells 210 may be formed of individual distinct pieces of padding material. In other embodiments, the cells 210 are formed by stitching a polygon pattern into the outer shell 300 to create polygon shaped cells 210 in the included padding 200. In some embodiments, one or more cells 210 form the shape of an icon, such as a graphic logo or symbol.

To use the wearable sleeves 10 generally consistent with the representative embodiment shown in FIGS. 1-6, the user slides the wearable sleeve 10 onto his or her arm such that the padding 200 is disposed on the lateral side of the forearm A. The user may then rest his or her arm A on a surface, such as a table, an armrest, or an airplane tray-table. The user may then rest his or her head onto the padding 200 portion of the wearable sleeve 100.

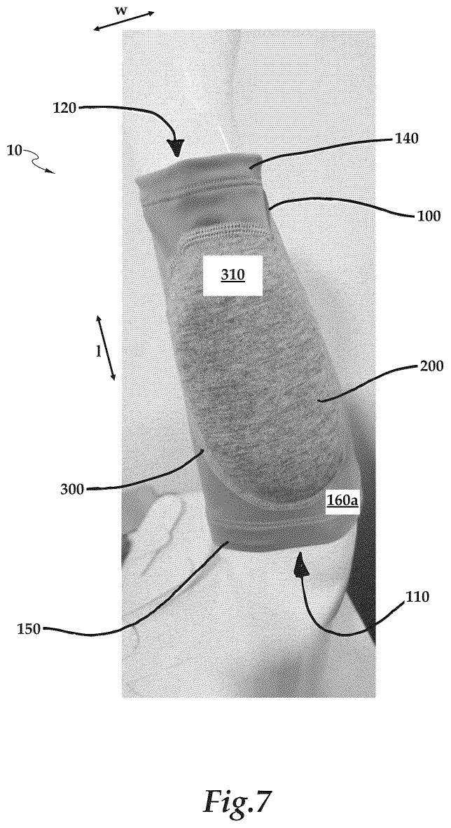

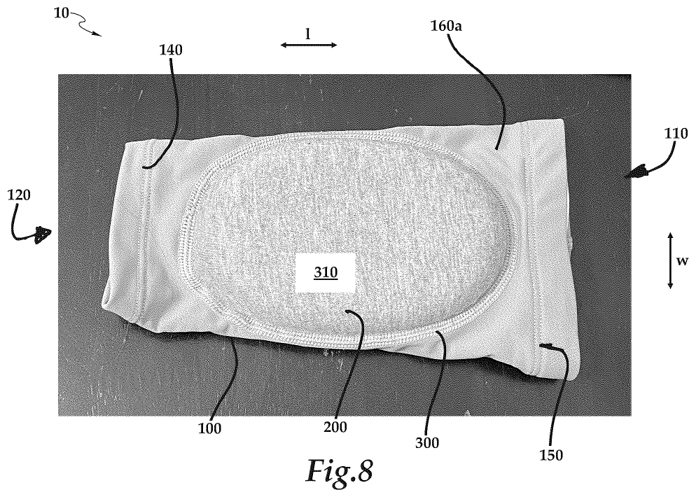

As shown representatively in FIGS. 7-8, wearable sleeves 10 may be configured to be worn on a forearm A. Generally, these embodiments are similar to those described above with respect to FIGS. 1-6, except the flexible sleeve 100 is sized and shaped to truncate at the elbow joint of the user. As such, embodiments consistent with that shown in FIGS. 7-8 may not include an upper arm section 170.

To use the wearable sleeves 10 generally consistent with the representative embodiment shown in FIGS. 7-8, the use slides the wearable sleeve 10 onto his or her arm such that the padding 200 is disposed on the lateral side of the forearm portion of the arm A. The user may then rest his or her arm A on a surface, such as a table, an armrest, or an airplane tray-table. The user may then rest his or her head onto the padding 200 portion of the wearable sleeve 100.

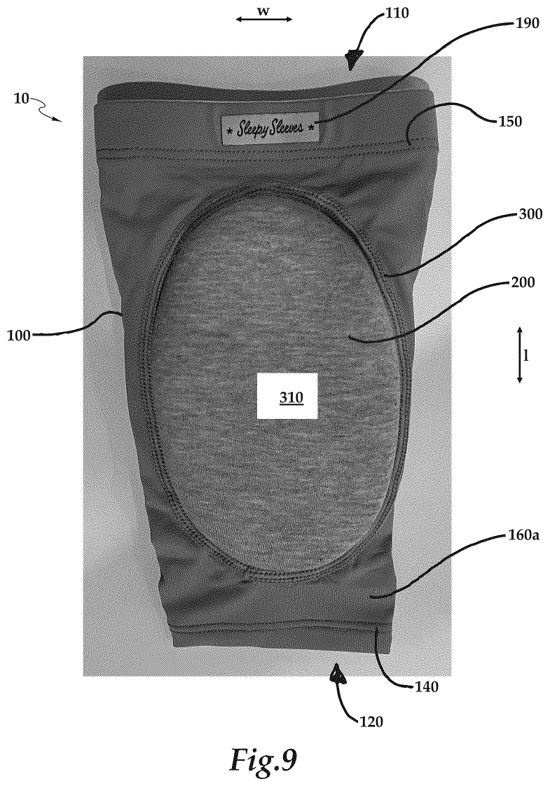

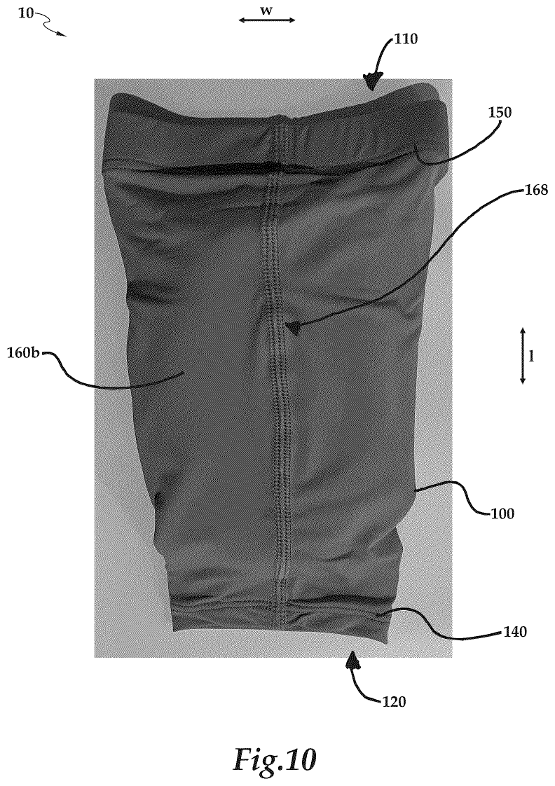

As shown representatively in FIGS. 9-10, a wearable sleeve 10 consistent with the present disclosure may include a flexible sleeve 100, padding 200 disposed on a first side of the flexible sleeve 100, and a seam 168 disposed generally from the bicep end 110 to the cuff end 120. The seam 168 in some embodiments may be disposed substantially opposite the padding 200.

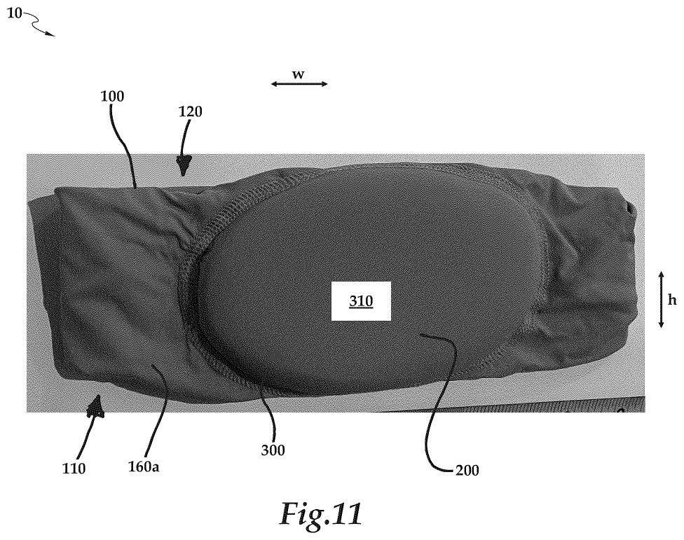

Referring now to FIGS. 11-14, wearable bands 10 may be configured to be worn on the head H, such as on the forehead.

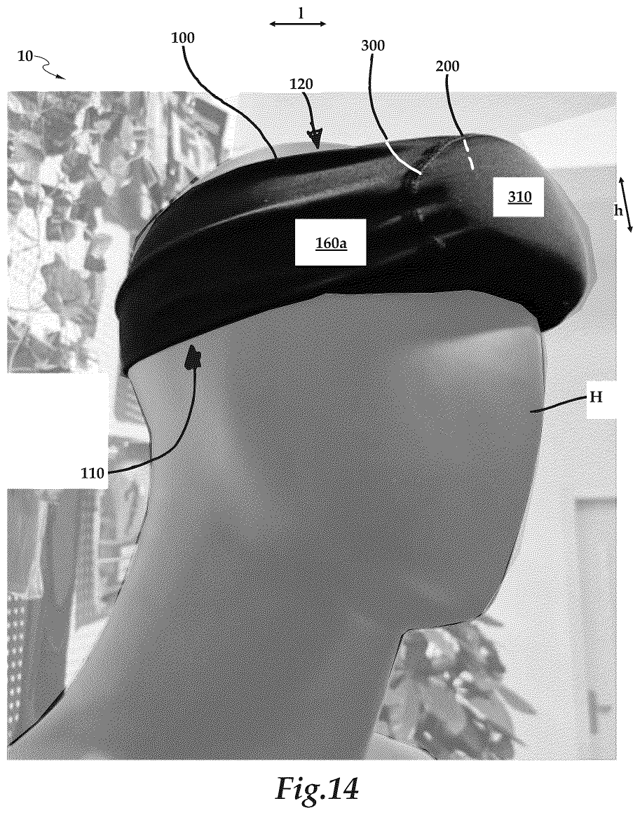

The flexible band 100 may be similar to the flexible sleeves 100 described above, except that the height h of the flexible band 100 of the head-worn wearable bands 10 corresponds to the length/of the flexible sleeves 100 described above. The flexible band 100 also has a width w (FIGS. 11-14) and a length/(FIG. 14), or alternatively a circumference, such as a circumference or maximum circumference at the lower end 110.

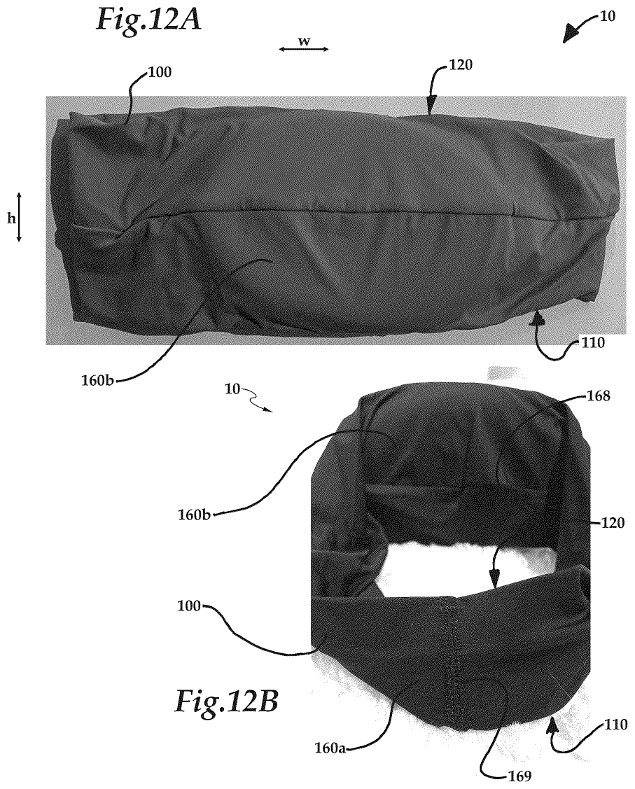

Referring now specifically to FIGS. 12A-12B, the wearable bands 10 may include a seam 168. For example and without limitation, the seam 168 may be disposed on an inside surface 160b of the flexible band 100, such that the seam 168 is not visible to an observer when adorned by the user. The seam 168 may run substantially parallel (e.g., parallel) to the width w of the flexible band 100, as is shown in FIGS. 12A-12B, or alternatively may run anti-parallel to the width w of the flexible band 100.

In some embodiments, such as those consistent with FIG. 12B, the wearable bands 10 may include a second seam 169. The second seam 169, when present, may be disposed antiparallel, for example orthogonal, to the seam 168 (e.g., substantially orthogonal to the width w of the flexible band 100). In some embodiments, the second seam 169 is disposed on the outside surface 160a of the flexible band 100. In other embodiments, the second seam 169 is disposed on the inner surface 160b of the flexible band 100. In still other embodiments, the second seam 169 is disposed on both the outside surface 160a and the inner surface 160b of the flexible band 100.

Although the term “band” is used to describe these head-worn embodiments, one of skill in the art will readily recognize that wearable bands 10 consistent with the embodiments shown in FIGS. 11-16 may not strictly be bands, but instead may be caps (e.g., dome-shaped caps) wherein the top end 120 is closed while the bottom end 110 is open.

In some embodiments, the padding 200 of head-worn stretchable bands 10 is generally disposed on the front outer surface 160a of the stretchable band 100. In other embodiments, the padding 200 is generally disposed on the rear outer surface 160b. In still other embodiments, the padding 200 is generally disposed on one or both sides of the outer surface 160. In some embodiments, more than one padding area 200 is disposed on the outer surface 160 of the head-worn flexible sleeve 100. In some embodiments, stitching 171 may be disposed proximal to the padding 200 to limit movement of the padding 200 relative to the flexible sleeve 100. To use the wearable band 10 generally consistent with the representative embodiment shown in FIGS. 11-16, the user slides the wearable band 10 onto his or her forehead H such that the padding 200 is disposed on forehead side, the rear side, or one of the lateral crown portions of the head H. The user may then rest his or her head H onto a surface such as a table, an armrest, or an airline tray-table, plane wall, person, wall W, vehicle interior surface, train interior surface, desk D, etc.

Generally, wearable pillows 10 of the present disclosure may be configured to be worn on a wearer's arm A, on a wearer's head H, or on a wearer's leg L, and include a tube-shaped body 100 configured for placement about a portion of a user's body; and a padding compartment 300 disposed on at least a portion of the tube-shaped body 100. Padding 200 is disposed within the padding compartment 300; the padding 200 may be secured (e.g., permanently secured) within the padding compartment 300, or alternatively may be removable from the padding compartment 300.

The circumference (πd₁₁₀ Or πd₁₂₀) of an opening of one end 110/120 of the tube-shaped body 100 may in some embodiments be about equal to a length of the tube-shaped body 100. In other embodiments, the circumference (πd₁₁₀ Or πd₁₂₀) of an opening of one end of the tube-shaped body 100 may be about two thirds of the length of the tube-shaped body 100.

The padding compartment 300 may in some embodiments comprise a polygonal shape, such as a circle, an oval, a triangle, a rectangle, a pentagon, a hexagon, a heptagon, an octagon, etc.

In some embodiments, the width of the padding compartment 300 is about equal to the length d₁₂₀ of a smaller end 120 of the tube-shaped body 100. In some embodiments, the length of the padding compartment 300 is about two thirds of the length of an inner seam length of the tube-shaped body 100.

In some embodiments, the padding compartment 300 is oriented generally parallel to a long side (e.g., length) of the flexible sleeve 100. In other embodiments, the padding compartment 300 is oriented at a predetermined offset angle relative to a longitudinal axis of the flexible sleeve 100.

The padding compartment 300 may be located in a center region of the flexible sleeve 100, for example approximately mid-way between one open end 110 of the flexible sleeve 100 and the opposite open end 120 of the flexible sleeve 100. In some embodiments, the padding compartment 300 is disposed about 2 cm to about 20 cm from a longitudinal edge of the flexible sleeve 100, for example about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, or about 20 cm from a longitudinal edge of the flexible sleeve 100.

In some embodiments, the padding compartment 300 is disposed about 2 cm to about 20 cm from a horizontal edge of the flexible sleeve 100, for example about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, or about 20 cm from a horizontal edge of the flexible sleeve 100.

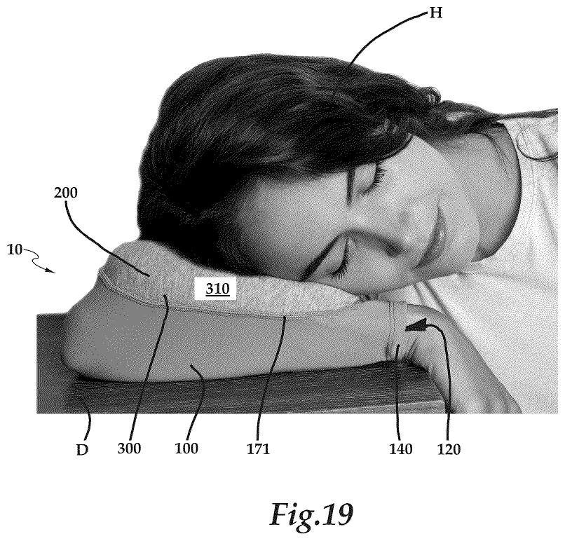

Referring now to FIG. 19, padded sleeves 10 consistent with the present disclosure may be used to support the head H of a user during rest or sleep, for example by sliding the flexible sleeve 100 over the arm A such that the padding 200 is disposed on the top side of the user's arm A. The head H can then be rested on the padding 200.

Alternatively, padded sleeves 10 consistent with the present disclosure may be used to support an infant during rest, bottle feeding, or nursing. In such embodiments (not shown), the padded sleeve 10 is slid onto the user's arm A such that the padding 200 is disposed on the top side of the arm A when the arm A is held in a cradling position. The infant can then rest comfortably on the padding 200 while napping, bottle feeding, or nursing.

The materials, methods, and examples described are illustrative in nature and not intended to be limiting. Accordingly, this disclosure should not be construed as limited to the illustrated embodiments. Other features and advantages of the disclosure will be apparent from the following detailed description and claims.

In the foregoing detailed description, numerous details, examples, and embodiments of the invention are described. However, it will be clear and apparent to one skilled in the art that the technology disclosed herein is not limited to the embodiments set forth and that the technology disclosed herein can be adapted for any of several applications. The drawings and specification describe various embodiments of the invention. Although specific terms are employed, the terms are used in a descriptive sense only and not for purposes of limitation. The invention has been described in detail with specific reference to the illustrated embodiments. It will be apparent, however, that various modifications and changes can be made within the spirit and scope of the invention as described in the specification and as defined in the appended claims.

1: A wearable arm sleeve comprising:
a flexible sleeve including a bicep end and a cuff end disposed opposite the bicep end; and
padding disposed on said flexible sleeve and covered by an outer shell having an outer surface.

2: The wearable arm sleeve of claim 1, wherein the flexible sleeve extends to an elbow of a wearer.

3: The wearable arm sleeve of claim 1, wherein the flexible sleeve comprises a stretchable fabric material.

4: The wearable arm sleeve of claim 1, wherein the flexible sleeve has a lengthwise stretchability of about 5% to less than 30%.

5: The wearable arm sleeve of claim 1, wherein the flexible sleeve has a width-wise stretchability of about 5% to less than 45%.

6: The wearable arm sleeve of claim 1, wherein the padding comprises a convex shape.

7: The wearable arm sleeve of claim 1, wherein the padding extends substantially the length of the sleeve.

8-11. (canceled)

12: The wearable arm sleeve of claim 1, wherein the padding comprises an inflatable air bladder and inflation port.

13-14. (canceled)

15: The wearable arm sleeve of claim 1 further comprising at least one opening in the flexible sleeve configured to promote air circulation against skin of a user.

16: The wearable arm sleeve of claim 1 further comprising at least one opening in the padding configured to promote air circulation against skin of a user.

17-18. (canceled)

19: A wearable head band comprising:
a flexible sleeve comprising a stretchable fabric material and including a neck end and a crown end disposed opposite the neck end; and
padding disposed on said flexible sleeve and covered by an outer shell having an outer surface,
wherein the flexible sleeve has a lengthwise stretchability of about 5% to less than 30%.

20-21. (canceled)

22: The wearable head sleeve of claim 19, wherein the flexible sleeve has a width-wise stretchability of about 5% to less than 45%.

23-24. (canceled)

25: The wearable head sleeve of claim 19, wherein the padding material comprises an inflatable air bladder and inflation port.

26-27. (canceled)

28: The wearable head sleeve of claim 19 further comprising at least one opening in the flexible sleeve configured to promote air circulation against skin of a wearer.

29: The wearable head sleeve of claim 19 further comprising at least one opening in the padding configured to promote air circulation against skin of a wearer.

30-31. (canceled)

32: A wearable pillow comprising:
a tube-shaped body configured for placement about a portion of a user's body; and
a padding compartment disposed on at least a portion of the tube-shaped body; and
padding disposed within the padding compartment,
wherein a circumference of an opening of one end of the tube-shaped body is about equal to a length of the tube-shaped body.

33-34. (canceled)

35: The wearable pillow of claim 32, wherein a circumference of one end of the tube-shaped body is about two thirds a length of the tube-shaped body.

36-37. (canceled)

38: The wearable pillow of claim 32, wherein a width of the padding compartment is about equal to a length of a smaller end of the tube-shaped body.

39-47. (canceled)

48: The wearable pillow of claim 32, wherein:
the tube-shaped body comprises a sheet of material capable of being formed into a tubular shape; and
the sheet of material includes a first mating feature disposed near one edge of the sheet of material, and a second mating feature disposed remotely from the first mating feature and configured to releasably mate with the first mating feature to form the tubular shape.

49. (canceled)

50: The wearable pillow of claim 48, wherein:
the first mating feature is selected from the group consisting of: a magnet, a snap stud, a snap socket, a button, a button hole, hook fastener, loop fastener, and a portion of a zipper; and
the second mating feature is complementary to the first mating feature and is selected from the group consisting of: a magnet, a snap socket, a snap stud, a button hole, a button, loop fastener, hook fastener, and a portion of a zipper.
